(19) United States
(12) Patent Application Publication
(10) Pub. No.: US 2023/0226361 A1
(43) Pub. Date: Jul. 20, 2023

(54) CARDIAC MAPPING TO EVALUATE IMPACT OF INTERVENTIONS

(71) Applicant: CARDIOINSIGHT TECHNOLOGIES INC., INDEPENDENCE, OH (US)

(72) Inventors: Qing LOU, SOLON, OH (US); Qingguo ZENG, SOLON, OH (US); TIMOTHY G. LASKE, SHOREVIEW, MN (US)

(21) Appl. No.: 18/057,454

(22) Filed: Nov. 21, 2022

Related U.S. Application Data
(60) Provisional application No. 63/299,591, filed on Jan. 14, 2022.

(51) Int. Cl. A61N 1/365; A61M 5/172; A61B 18/00

(52) U.S. Cl. CPC A61N 1/36592; A61B 18/00; A61M 5/1723; A61B 2018/00351; A61B 2018/00577; A61B 2018/00642; A61B 2018/00839; A61M 2210/125; A61M 2230/04

(57) ABSTRACT

A computer-implemented method includes accessing electrophysiological data and generating an electroanatomic map for a surface of interest based on the electrophysiological data acquired during or after application of a first intervention to temporarily perturb electrical properties of a region of interest on or within the patient’s heart. The method also includes determining changes in the map or information derived from the map responsive to application of a first intervention. The first intervention can include including applying a non-lethal energy and/or a bioactive agent to induce or inhibit conduction of electrical activity for the region of interest. The method also includes controlling a second intervention to permanently alter the electrical properties of the region of interest based on the determination indicating a desired change in cardiac electrical activity responsive to the first intervention.

### CROSS REFERENCE TO RELATED APPLICATION

This application claims the benefit of U.S. Provisional Pat. Application No. 63/299591, filed 14 Jan. 2022, which is incorporated herein by reference in its entirety.

### FIELD

The present technology is generally related to generating one or more cardiac maps to evaluate the impact of one or more interventions.

### BACKGROUND

Electrophysiology procedures are used to analyze, diagnose and/or treat cardiac electrical activities. Electrophysiology procedures usually take place in an electrophysiology (EP) lab or a catheterization (Cath) lab at a hospital or other medical facility. For example, an EP mapping procedure can be performed in an invasive procedure in which one or more electrode catheters are placed in or on the heart to measure electrophysiology signals. In an additional or alternative example, the EP mapping procedure may be performed using a non-invasive arrangement of electrodes distributed across an outer surface of the patient’s body (e.g., on the thorax). In a given EP procedure, the user is tasked with entering respective inputs to control system parameters, acquire and process measured data as well as to determine and control how to generate relevant maps.

### SUMMARY

The techniques of this disclosure generally relate to generating cardiac maps to evaluate the impact of one or more interventions.

In one aspect, the present disclosure provides a method that includes applying a first intervention to perturb electrical properties of a region of interest on or within a patient’s heart during a perturbation interval that includes at least a portion of one or more cardiac cycles. The method also includes generating, by a computing device comprising a processor, an electroanatomic map for a surface of interest based on cardiac electrophysiological data representing cardiac electrophysiological signals over a time interval that includes the perturbation interval. The method also includes evaluating, by the computing device, the map for at least the time interval to determine changes in cardiac electrical activity responsive to the first intervention. The method also includes controlling a second intervention to permanently alter the electrical properties of the region of interest based on the determined changes in the cardiac electrical activity.

In yet another aspect, the disclosure provides a system to evaluate the impact of an intervention. The system includes non-transitory memory configured to store data and machine-readable instructions. The data includes electrophysiological data representing cardiac electrophysiological signals for a plurality of locations across a cardiac surface over time. One or more processors are adapted to access the memory and execute the instructions programmed to perform a method. The method performed by execution of the instructions includes generating an electroanatomic map for the cardiac surface based on the electrophysiological data acquired over time that includes a first time interval and at least one other time interval. One of the first or other time intervals can occur during or after (e.g., so as to be responsive to) application of a first intervention to temporarily perturb electrical properties of a region of interest on or within a patient’s heart. The method performed by execution of the instructions also includes determining changes in the map or in information derived from the map between the first time interval and the other time interval. The method also includes controlling a second intervention to permanently alter electrical properties of the region of interest based on the determined changes. In an example, the other time interval can occur before or after the first intervention.

In yet another aspect, the disclosure provides a computer-implemented method that includes accessing electrophysiological data and generating an electroanatomic map for a surface of interest based on the electrophysiological data acquired during or after application of a first intervention to temporarily perturb electrical properties of a region of interest on or within the patient’s heart. The method also includes determining changes in the map or information derived from the map responsive to application of a first intervention. The first intervention can include including applying a non-lethal energy and/or a bioactive agent to induce or inhibit conduction of electrical activity for the region of interest. The method also includes controlling a second intervention to permanently alter the electrical properties of the region of interest based on the determination indicating a desired change in cardiac electrical activity responsive to the first intervention.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram of an example system that can be implemented for measuring and monitoring electrophysiological signals.

FIG. 2 is a block diagram of an example electrophysiological data analyzer that can be used in the system of FIG. 1.

FIG. 3 is a block diagram of an example feature calculator that can be used in the system of FIG. 1.

FIG. 4 is a flow diagram of an example method that can be implemented for generating one or more cardiac maps to evaluate cardiac electrical activity.

FIG. 5 is a flow diagram of an example method that can be implemented for evaluating impact of one or more interventions.

### DETAILED DESCRIPTION

This disclosure relates to mapping used to evaluate the impact of one or more interventions. For example, systems and methods disclosed herein can generate an electroanatomic map in real-time (or near real-time) in electrophysiology (EP) studies and procedures to visualize the impact of an intervention applied to a patient with respect to the patient’s cardiac electrical activity. As used herein, an intervention can refer to any act having an effect to alter electrical properties of a patient’s heart. In an example, an intervention includes a non-lethal perturbation of electrical properties of a region of interest on or within a patient’s heart, in which the perturbation of electrical properties is temporary (e.g., the perturbation of electrical properties exists during application of the intervention or are otherwise reversible). In another example, an intervention is applied to permanently alter the electrical properties of the region of interest.

An intervention can be applied in one or more modes or manners, such as by applying an energy, a bioactive agent or a combination of energy(ies) and agents to alter the electrical properties of the region of interest. For example, the intervention can be applied to induce or inhibit conductivity of electrical signals for the region of interest. Also, the intervention can be applied directly to the region of interest of the patient’s heart or indirectly to the region of interest through another part of the patient’s heart or elsewhere in the patient’s body. One or more interventional devices can be configured to apply the energy and/or bioactive agent to effect the perturbation at the region of interest on or within the patient’s heart. It is to be understood that the systems and methods (e.g., a computer-implemented method) can be implemented without applying the intervention, such as by configuring such systems and methods are configured to detect changes in the measured cardiac electrical activity. That is, interventional devices as well as methods of applying interventions can be separately implemented apart from the systems and methods disclosed herein.

As an example, during EP studies and procedures in the absence of the systems and methods disclosed herein, electrophysiologists may be uncertain on where to ablate for certain arrhythmias, such as persistent atrial fibrillation (AF). This is because the feedback that electrophysiologists can obtain from using existing technologies is limited. By implementing systems and methods described herein in combination with (or integrated with) a navigation/EP system, additional guidance and feedback can be provided to help improve outcomes for cardiac ablation and other interventions. For example, a computing device can be programmed to detect changes in electrical activity (e.g., changes in cardiac rhythms, cycle length and/or other signal characteristics) responsive to application of an intervention to alter electrical properties of a region of interest. A second intervention can be applied to the same or different region of interest based on the detected changes in electrical activity. The second intervention can be permanent or temporary, such as can be determined based on the detected changes in electrical activity responsive to applying the first intervention. For example, the detected changes can be determined by comparing (e.g., computing a difference between) measured electrical activity before or after the first intervention and the electrical activity responsive to (e.g., during or immediately after) the first intervention. As a result of using the systems and methods, improved treatment strategies can be determined and a desired therapeutic effect can be achieved more efficiently, thereby improving patient outcomes and user experience.

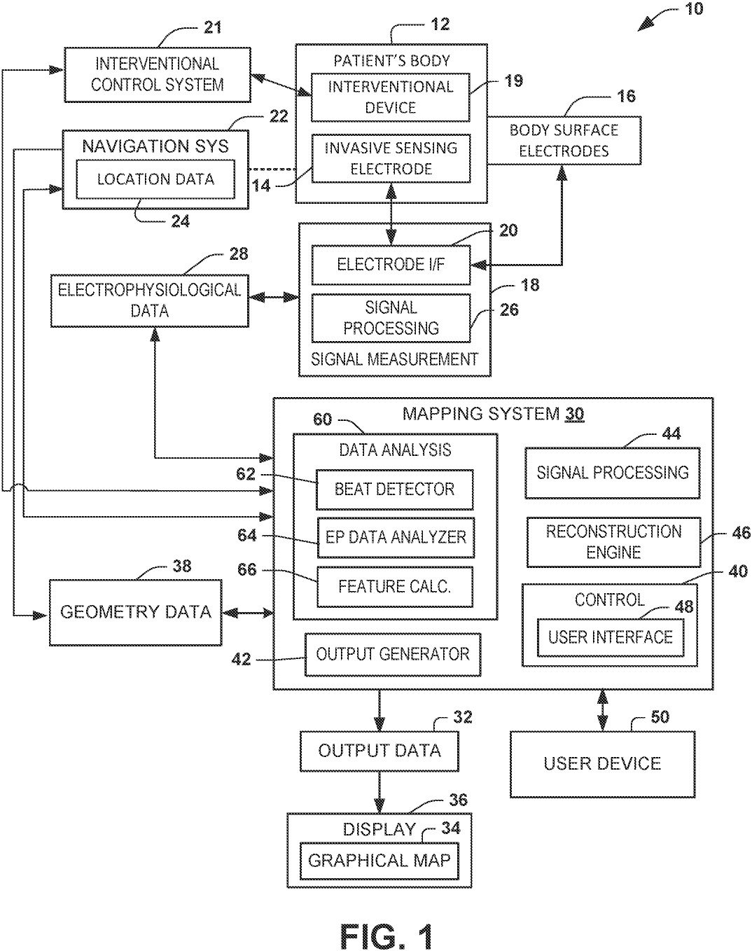

FIG. 1 depicts an example of a system 10 for evaluating the impact of an intervention based on monitoring and mapping electrophysiological signals measured from a patient 12. The system 10 can include one or more invasive sensing electrodes 14. The system 10 can additionally, or alternatively, include body surface electrodes 16. For purposes of consistency, the following description describes the system as having both invasive sensing electrodes 14 and body surface electrodes 16. However, in other examples, the system 10 may include only invasive electrodes 14 or only body surface electrodes 16. The respective electrodes 14, 16 are coupled to a signal measurement device 18.

As an example, each of the electrodes 14, 16 is coupled to the signal measurement device (or subsystem) 18 through a respective electrically conductive channel (e.g., including electrically insulated wires and/or traces) to communicate electrophysiological signals measured from the patient’s body. The channels for respective electrodes 14 and 16 can also include an arrangement of connectors configured to couple to respective connectors (e.g., male and female connectors) of an electrode interface 20 of the measurement device 18 as well as amplifiers, filters and the like. In other examples, the electrodes 14, 16 may be coupled to the electrode interface 20 through other forms of communication (e.g., optical fiber or wireless leads). The electrode interface 20 can measure unipolar, bipolar or a combination of unipolar and bipolar electrophysiological signals depending on the configuration of the measurement device 18 and processing of the signals measured by the electrodes 14 and 16.

As an example, the one or more invasive sensing electrodes 14 can be coupled to or otherwise carried by a device, such as an interventional device 19. In such example, the interventional device 19 can be implemented as an electrophysiology probe or catheter to which one or more sensing electrodes 14 are coupled that is moveable within the patient’s body 12, such that the position of the device 19 and associated electrode(s) 14 can vary over time. For example, a cardiac catheter can be inserted into a femoral vein (or other known entry point) and advanced to a position within the patient’s heart so the sensing electrode(s) 14 are adapted to measure electrophysiological signals within the heart. Alternatively, the electrode(s) 14 can be configured to measure electrophysiological signals on an outer surface of the patient’s heart. Thus, the signals measured by the invasive sensing electrodes 14 depend on where the probe is positioned within the patient’s body 12. The interventional device 19 may be moved manually, robotically assisted or fully robotically to control where the sensing electrode 14 is position. Alternatively, the invasive sensing electrode(s) 14 can be implemented on a device that is separate from the interventional device 19 (e.g., a catheter or probe).

The interventional device 19 is configured to apply an intervention to perturb electrical properties of a region of interest on or within a patient’s heart. For example, the interventional device 19 includes an arrangement of components configured to apply the intervention such as by delivering an energy and/or a bioactive agent to the patient. The applied intervention thus can operate to induce or inhibit conduction of electrical signals for the region of interest. As described herein, the intervention (e.g., applied by interventional device 19) can be non-lethal or lethal to the region of interest where the intervention is applied, such as depending on a duration that the effects of the intervention last.

As an example, the applied intervention can be non-lethal with respect to the ROI if the effects on the electrical properties of the ROI are temporary (e.g., transitory). Thus, the terms non-lethal and temporary can be used interchangeably to describe such intervention. A temporary intervention can be used to facilitate exploration of one or more desired target treatment sites. The effects can be considered temporary because the alteration of electrical properties at the ROI end responsive to or after the application of the intervention is stopped. The intervention can also be considered temporary if a reversal procedure can be implemented (e.g., by the interventional device 19 or another device) to remove or reverse the alteration of electrical properties at the ROI caused by the intervention. Examples of non-lethal (e.g., temporary) energy interventions that can be applied by the interventional device 19 include electrical stimulation (e.g., localized defibrillation, sub-threshold pacing, low energy stimulation), cryomapping, low amplitude pulsed-field ablation (PFA) or reversible electroporation. Examples of non-lethal (e.g., temporary) bioactive agent interventions that can be applied by the interventional device 19 include attaching temporary implants and/or injecting chemicals (e.g., alcohol, Lumason, Amiodarone, electrolytes or other chemicals) to induce or inhibit electrical conduction in the region of interest.

As a further example, an applied intervention can be considered permanent with respect to the region of interest if the effects on the electrical properties of the region of interest are permanent, such as being cytotoxic or irreversible to the tissue. Thus, the terms lethal, irreversible and permanent can be used interchangeably to describe such intervention. As an example, a permanent intervention lasts an extended duration, such as the remainder of the patient’s natural life. A permanent (e.g., irreversible) intervention can be applied to effect a permanent perturbation of electrical properties of the region of interest, such as by modifying the physiology of cardiac tissue. Examples of permanent (e.g., irreversible) interventions that can be applied by the interventional device 19 include ablation (e.g., RF ablation, high amplitude PFA, laser ablation, chemical ablation, surgery) to the region of interest.

In some examples, the same interventional device 19 are used for application of both temporary and permanent interventions. In other examples, different interventional devices 19 are used, in which a first device is used to apply a temporary intervention and a second device is used to apply a permanent intervention. Thus, each interventional device 19 can be implemented in a variety of different ways depending on the type of intervention to be implemented. Some examples of interventional devices include a laser applicator, a signal generator and one or more electrodes on an ablation catheter of various shapes (e.g., focal, linear, circular, etc.), a needle, syringe, a scalpel, a cryoablation tool, etc. As a further example, the interventional device 19 can be implemented as the Arctic Front cryoablation catheter system, the DiamondTemp ablation system, or other ablation products commercially available from Medtronic plc as well as other companies.

In some examples, such as where the interventional device 19 is configured to apply some form of energy, the system 10 can also include an interventional control system 21. The control system 21 is configured to control the operation of the interventional device 19, such as by setting operating parameters and supplying electrical power. As an example, the control system 21 includes hardware and/or software configured to control parameters of the energy being supplied to the device 19 for applying a corresponding intervention to the patient’s body to alter electrical properties of a region of interest of the heart. For the example of an electrical stimulation intervention, the parameters can include energy level (e.g., current and voltage), pulse width, duty cycle, and repetition rate. For the example of a laser intervention the parameters can include energy level, duration, wavelength and repetition rate. For the example of an RF ablation intervention the parameters can include energy level (e.g., current and voltage), duration, and repetition rate. For the example of a pulsed field ablation intervention, the parameters can include energy level (e.g., current and voltage), waveform composition and duration. Other parameters can be used and configured according to the type of interventional device, the intervention being applied and the anatomic location where the intervention is being applied. The parameters will depend on the type of interventional device 19, and the parameters can determine whether the intervention being applied is temporary or permanent. The parameters can remain fixed during application of a respective intervention or the control system 21 can vary one or more parameters during the application of the intervention.

The control system 21 can set the parameters and apply an intervention based on automatic, manual (e.g., user input) or a combination of automatic and manual (e.g., semiautomatic controls). One or more sensors (not shown) of the device 19 can also communicate sensor information (e.g., feedback) back to the control system 21. The sensor information can describe a sensed condition of the interventional device 19 and/or the tissue to which the intervention is being applied. The control system 21 can also be coupled to a mapping system 30, such as to receive instructions, such as commands (e.g., to set operating parameters) or to trigger the interventional device to apply the intervention. The control system 21 can also provide interventional data to the mapping system, such as describing parameters used for application of the intervention and a timestamp describing when the respective intervention is applied.

The system 10 can also include a navigation system 22 configured to localize the spatial position of the interventional device 19 and the sensing electrode(s) 14. The spatial position of the electrode 14 (and/or associated interventional device 19) can be stored in memory as location data 24. The location data 24 can represent a three-dimensional spatial position (e.g., spatial coordinates) and orientation of the electrode(s) 14 and/or the interventional device 19. Alternatively, the location data 24 can represent the location of a location sensor or other known location on the probe carrying the electrode(s), and the spatial location of each sensing electrode 14 and/or interventional device can be derived readily from the location data 24. In examples where the electrode 14 and interventional device 19 are integrated in a single device, the same location data 24 can represent the spatial position of both. In examples where the electrode 14 and interventional device 19 are implemented in separate devices, separate location data 24 can be generated to represent the spatial position of each.

The location data 24 can be with respect to the patient’s body or a coordinate system of the navigation system 22. For example, the spatial location of the invasive sensing electrode 14 and/or interventional device 19, which is described by or derived from the location data 24, can be registered with respect to anatomical geometry of the patient’s body 12. The registration can be repeated in response to detecting changes in the location data as the electrode is moved within the patient’s body. In some examples, the navigation system 22 can also generate the location data 24 to include the location of one or more of the non-invasive electrodes 16, which are distributed across an outer surface of the patient’s body (e.g., on the thorax). For example, the interventional device 19, the sensing electrode(s) 14 and/or body surface electrodes 16 can be sensorized (e.g., include sensors mounted located at known locations) to enable the navigation system 22 to track respective positions and orientation in real time.

Useful examples of the navigation system 22 include the STEALTH STATION navigation system (commercially available from Medtronic), the CARTO XP EP navigation system (commercially available from Biosense-Webster) and the ENSITE NAVX visualization and navigation technology (commercially available from St. Jude Medical); although other navigations systems could be used to provide the navigation data representative of the spatial position for the invasive electrode 14 and associated probe. Another example of a navigation system that can be utilized to localize the position of the invasive electrodes is disclosed in U.S. Pat. No. 10,323,922, issued Jun. 18, 2019 Aug. 29, 2014, and entitled LOCALIZATION AND TRACKING OF AN OBJECT, which is incorporated herein by reference. For example, a probe (e.g., catheter) can include one or more electrodes 14 disposed at known locations with respect to the probe. The probe can be used to position each such electrode 14 with respect to the heart and the navigation system 22 can determine corresponding three-dimensional coordinates for the electrode(s) 14 that is represented by the location data 24.

The number and placement of invasive electrodes 14 can vary depending upon the type of catheter or other device to which the electrodes are coupled. In a further example, the invasive electrode(s) can be contact electrodes that measure signals from a surface of an object that the electrode physically engages or contacts. Alternatively, the invasive electrode(s) 14 can be non-contact electrodes that measure signals from a surface of an object while the electrode is spatially apart from (e.g., no physical contact between the electrode and the surface being measured). Such electrodes thus can be used to perform mapping from the body surface or from within a cardiac chamber.

The body surface electrodes 16 include a distributed arrangement of multiple electrodes (e.g., about 50, 100, 250 or more sensors) positioned on an outer surface of the patient’s body 12. In an example, the body surface electrodes 16 are distributed completely around the thorax, such as can be mounted to a wearable garment (e.g., vest) in which each of the electrodes has a known location in a given coordinate system. For example, body surface electrodes 16 can be implemented as a non-invasive type of sensor apparatus as disclosed in U.S. Pat. Publication No. 2013/0281814, entitled Multi-Layered Sensor Apparatus. Other configurations and numbers of body surface electrodes 16 could be utilized in other examples.

As described above, the electrode interface 20 has respective inputs coupled to each of the electrodes 14 and 16. The signal measurement device 18 can also include signal processing circuitry and/or software function 26 configured to process electrical signals measured by the respective electrodes 14, 16. The signal processing circuitry 26 can be implemented as hardware and/or software, such as including a digital signal processor and other processing circuitry and machine readable instructions (executable by a processor) configured to remove noise (e.g., line noise) and convert the received signals into a desired format for storing the measured electrophysiological signals as electrophysiological data 28. The signal processing circuitry 26 can also add channel information (e.g., to specify electrode number or location), add timestamps (e.g., to specify the time or each measurement sample) or perform other signal processing functions that may be desired. The electrophysiological data 28 thus can include signal measurement values for each sample, including signal morphology, as well as additional information, such as time stamps and channel information. In an example, the signal processing circuitry 26 can extract signal morphology features, such as cycle length, dominant frequency, waveform geometry the like, and store such extracted signal features with the electrophysiological data 28.

The system 10 includes one or more processors configured to access memory that stores data. The processor(s) can access and execute instructions corresponding to the functions and methods implemented by the mapping system 30. The mapping system 30 thus includes instructions executable by the one or more processors of the computer device to perform the functions described herein. In the example of FIG. 1, the mapping system includes an output generator 42, signal processing function 44, a reconstruction engine 46 and data analysis function 60. The mapping system 30 also includes a control function 40 configured to control one or more processing, analysis and mapping functions implemented by the system 30.

In the example of FIG. 1, the reconstruction engine 46 (e.g., instructions executable by one or more processors) is programmed to compute reconstructed electrophysiological signals for locations on a surface of interest within the patient’s body 12. The reconstructed electrophysiological signals provide an electroanatomic map for the surface of interest. In one example, the reconstruction engine 46 computes the reconstructed signals (e.g., as electrical potentials) on the surface of interest by a processor executing machine-readable instructions (e.g., an algorithm) programmed to reconstruct electrical signals spatially and temporally on to the surface of interest based on the electrophysiological data 28 and the geometry data 38. As described herein, the geometry data 38 includes three-dimensional spatial information representing the surface (or surfaces) of interest describing a surface on to which reconstructed signals are computed (by engine 46) co-registered with respective locations where electrophysiological measurements are made (e.g., by the electrodes 14 and/or 16). The reconstruction engine 46 can calculate the reconstructed electrical signals on the surface of interest for one or more surfaces of interest over one or more time intervals, including one or more cardiac cycles. The time interval(s) may be selected through a user interface 48 in response to a user input entered by a user device 50 either locally or from a remote location (e.g., mouse, keyboard, touchscreen interface, gesture interface or the like). Alternatively, the time interval(s) can be selected automatically by the data analysis function 60.

As a further example, where the EP data 28 includes electrical signals measured by the body surface electrodes 16, the reconstruction engine 46 includes code programmed to implement the method of fundamental solutions (MFS). The reconstruction engine 46 thus employs the MFS to solve an inverse problem for computing reconstructed electrical signals on the surface of interest based on the EP data 28 and the geometry data. MFS includes a mathematical representation that spatially relates an influence of the electrophysiological signals measured on the outer surface of the patient’s body and the electrophysiological signals measured within the patient’s body to the electrophysiological signals on the surface of interest. In an example, the MFS method can implement MFS ECGI similar to that disclosed in U.S. Pat. No. 7,983,743, which is incorporated herein by reference, and further modified to utilize the electrophysiological data that includes measurements from both the invasive and non-invasive electrodes 14 and 16. Other useful examples of inverse algorithms that can be implemented by the reconstruction engine 46 to reconstruct include the boundary element method (BEM), such as disclosed in U.S. Pat. Nos. 6,772,004, and 9,980,660, each of which is incorporated herein by reference. The reconstruction engine 46 further may employ a regularization technique (e.g., Tikhonov regularization) to estimate values for the reconstructed electrical signals on the surface of interest.

The output generator 42 is programmed to generate output data 32 that can be rendered as a graphical map 34 on the display 36 to graphically visualize EP signals on a surface of interest. For example, the output generator 42 is programmed to generate an EP map based on the reconstructed signals (generated by reconstruction engine 46) and geometry data 38. As mentioned, reconstructed electrical signals can be derived from non-invasively measured signals (by electrodes 16), from invasively measured signals (by electrodes 14) or from a combination of non-invasively and invasively measured signals. By using EP data from both non-invasively and invasively measured signals to generate a respective map 34, the respective map can more accurately represent cardiac electrophysiological signals on the surface of interest.

As disclosed herein, the surface of interest may be an epicardial surface, an endocardial surface, a combination of an epicardial or endocardial surfaces or a full three dimensional rendering of the heart tissue, including epicardial, endocardial and transmural myocardium throughout the heart. Additionally, or alternatively, the surface of interest can be a cardiac envelope, such as a virtual surface residing between the center of a patient’s heart and the body surface where the electrodes are positioned. The surface of interest may encompass the entire cardiac surface or one or more regions (epicardial or endocardial) of interest. The output generator 42 thus is configured to provide the output data 32 to the display 36 to visualize one or more electrocardiographic maps 34 as well as other electrical information derived from the EP data 28 and geometry data 38. The output generator 42 can also provide information in other display formats to provide guidance to the user representative of and/or derived from electrical activity that may be measured by any combination of the electrodes 14 and 16. For example, the mapping system 30 may further use the output generator 42 to provide guidance to help a user move the invasive electrode 14 (or other interventional device) to a location of interest (e.g., on or near a region of interest of the heart) based on the electrophysiological data 28 and the geometry data 38.

The geometry data 38 includes electrode geometry data and anatomical geometry data. The electrode geometry data represents spatial locations of respective body surface electrodes 16 and invasive electrode 14 in three-dimensional space. The anatomical geometry data represents spatial geometry of the surface of interest of the patient in three-dimensional space. The mapping system 30 can further programmed to co-register the electrode geometry data, the position of the interventional device 19 and the anatomical geometry data in a common coordinate system to provide the geometry data 38. The spatial registration function can implement one or more transforms to align spatially respective data sets for location of the invasive electrodes 14, the location of the interventional device 19, the location of the body surface electrodes 16 as well as the anatomical geometry for the surface of interest.

As an example, the navigation system 22 generates location data 24 to represent the spatial location of the invasive electrodes 14 in a given coordinate system (e.g., of the navigation system), which may be different from the coordinate system in which the anatomical geometry data is generated. The anatomical geometry data can be derived from imaging data acquired by a three-dimensional medical imaging modality. The medical imaging data can be generated for the patient’s body using a medical imaging modality, such as single or multi-plane x-ray, computed tomography (CT), magnetic resonance imaging (MRI), ultrasound, positron emission tomography (PET), single-photon emission computed tomography (SPECT) and the like. The electrode locations and locations of the surface (or surfaces) of interest can be identified in a respective coordinate system of the acquired images through appropriate image processing, including extraction and segmentation. For instance, segmented image data can be converted into a two-dimensional or three-dimensional graphical representation that includes the volume of interest for the patient. Appropriate anatomical or other landmarks, including locations the electrode 14, can be identified in the geometry data 38 to facilitate spatial registration of the electrophysiological data 28. The identification of such landmarks can be done manually (e.g., by a person via image editing software) or automatically (e.g., via image processing techniques). In one example, an anatomical model can be constructed based on imaging data obtained (e.g., by a medical imaging modality) for the patient to provide spatial coordinates for points across the surface of interest. In some cases, in which the electrodes 16 are positioned on the patient’s body when the medical image is acquired, spatial coordinates can be provided for the locations where the body surface electrodes 16 are positioned on the outer surface of the patient’s body.

In another example, the location of the body surface electrodes 16 can be acquired by a digitizer, manual measurements or another non-imaging based technique, such as including being obtained by the navigation system 22 and included as part of the location data 24. The spatial registration function can provide the geometry data 38 to include the location information for the electrodes 14 and 16 as well as the anatomical geometry all spatially aligned in the common coordinate system. Because the location of the invasive electrode 14 can be moved within the patient’s body 12, the corresponding location data 24 can be updated (e.g., in real-time or near-real time) to reflect the current spatial location where the invasive electrical measurement is obtained. Thus, the navigation system 22 can further be programmed to update the geometry data 38 in response to detecting change in the location data 24 for one or more electrodes 14. The location data 24 can also include a time stamp so that the mapping system 30 can programmatically link (e.g., synchronize) a given time instance of the geometry data 38, which includes locations of the electrode 14 and interventional device 19, with respect to samples of the electrophysiological signals that are measured.

As a further example, the control function 40 can include instructions to control the output generator 42 to instruct the user to move the interventional device 19 to a target location (e.g., by generating a notification to position the catheter or probe) based upon detected changes in rhythms or other signal features, which can be automatically detected. This can help improve the fidelity of mapping and/or improve delivery of a desired intervention by interventional device 19 (e.g., therapy, ablation, CRT, application of a bioactive agent or other intervention) at one or more target sites.

In a further example, the control function 40 is configured to control the output generator 42 and/or signal processing function 44 based on the data analysis function 60. In the example of FIG. 1, the data analysis function 60 includes a beat detector function 62, an EP data analyzer function 64 and a feature calculator function 66.

The beat detector function 62 is programmed to analyze the EP data 28 and define a plurality of heartbeat intervals for one or more respective EP signals. The beat detector 62 can detect heartbeats in one signal type or combination of such different signals that can be measured or generated by the system 10. In one example, the EP signals can correspond to a signal measured by one or more of the body surface electrodes 16. In another example, the EP signal(s) used by the beat detector 62 can include signals measured by the invasive electrode 14. In yet another example, the EP signals used by the beat detector 62 can be reconstructed EP signals (e.g., generated by reconstruction engine 46). Various existing heartbeat detection algorithms could be implemented by the beat detector 62, for example the Pan-Tomkins, or modified versions on the intracardiac EP signals. The beat detector 62 can append start and/or stop time information to the EP data 28 for each detected heartbeat, such as by tagging respective beats (e.g., with metadata) in such data or otherwise storing information in memory to specify the respective heartbeats. The beat detector 62 can identify the heartbeat interval automatically, such as described above, or in response to a user input selecting one or more respective intervals of signals (e.g., on a graphical user interface).

In one example, the beat detector 62 is programmed to tag one or more heartbeat intervals responsive to application of an intervention by the interventional device 19 to alter electrical properties of a region of interest in or on the heart. For example, the control system 21 can provide intervention data indicating when the interventional device 19 applies a respective intervention, and the intervention data can also include parameter data describing the parameters associated with the applied intervention. The navigation system 22 can also provide the location data 24 to specify location and orientation of the interventional device, including during application of the intervention. Thus, for a respective intervention that is applied the beat detector 62 can tag or otherwise link the EP data 28 with the intervention data and location data 24 associated with the respective intervention.

The EP data analyzer 64 is programmed to analyze a portion of the EP data 28 defined by respective heartbeat intervals (e.g., as defined by beat detector 62) to determine one or more parameters associated with the respective beats of EP signals over one or more time intervals. The time intervals can be selected in response to user input or correspond to various time intervals for which the EP data 28 has been acquired and encompass the detected heartbeats. In one example, the time interval can include EP data that is measured responsive to a respective intervention, which can occur during or after application the respective intervention. The EP data analyzer 64 is configured to generate the parameters to represent a number of EP signals representative of cardiac electrical activity across the surface of interest. For example the surface of interest can be a cardiac surface of interest such as an endocardial surface, epicardial surface or surface. Alternatively, the surface of interest can be a virtual surface within a patient’s body. As mentioned, reconstruction engine 46 can reconstruct the measured EP signals onto the surface of interest. Thus in some examples, the data analyzer 64 can be applied to analyze a particular region (or subregion) of the surface of interest of the patient’s heart. Alternatively or additionally, the analysis can be implemented with respect to the entire surface of interest (e.g., the entire cardiac surface) for each of the respective heartbeat intervals.

For example, the EP data analyzer 64 can derive a number of signal parameters for the EP signals, which can include parameters for each heartbeat or parameters that describe signal characteristics over more than one heartbeat. The signal parameters can be low-level parameters that describe attributes of respective signal waveforms and may be extracted or derived directly from a given signal waveform. Examples of signal parameters include amplitude, slew rate, frequency components as well as morphological characteristics of one or more components of each heartbeat interval. Alternatively or additionally, the signal parameters can be derived from components of the body-surface or unipolar EP waveform, such as to describe one or more attributes of the P, Q, R, S, T waveform components, such as width of respective segments or intervals, amplitude, slope, number of peaks or parameters that describe a combination of two or more waveform components (e.g., QRS duration and/or morphology, R-R interval, P-P interval, QT duration and/or morphology, etc.). In a further example, each of the different waveform components can be parameterized by a number of signal and/or morphological parameters that can be stored in memory associated with the respective heartbeat interval and signal for which the interval is associated. Such parameters may be stored as part of the EP data 28 (e.g., as signal parameter metadata) or otherwise associated (or linked) with the EP data.

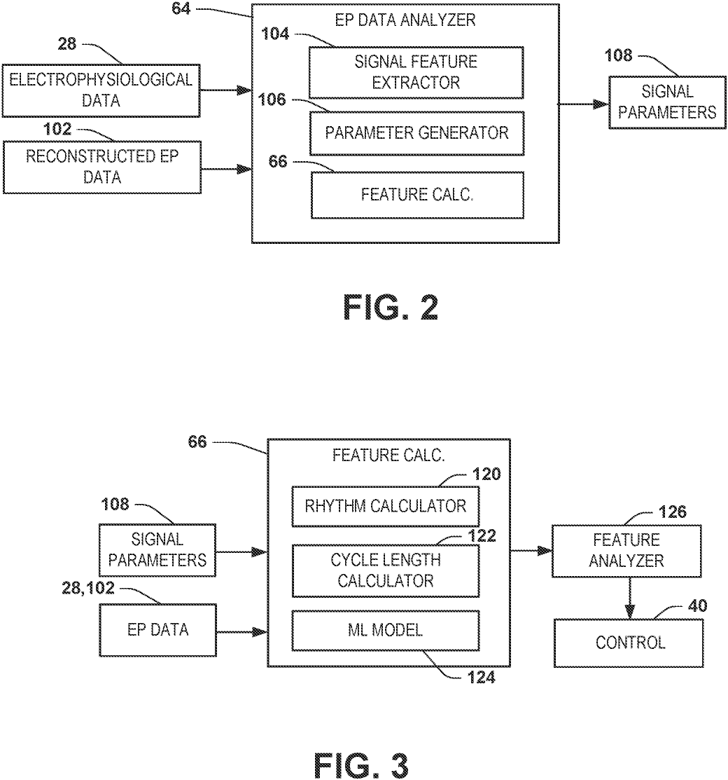

In an example, the EP data analyzer 64 can include a feature calculator 66 programmed to compute one or more signal features associated with the cardiac electrophysiological signals over at least a portion of a time interval, such as based on one or more parameters (e.g., determined by the EP data analyzer 64). The feature calculator 66 can be part of the EP data analyzer 64 (see, e.g., FIG. 2) or it can be implemented as separate program code as shown in FIG. 1. The feature calculator 66 can compute respective features of the EP signals distributed across a region of interest. The region of interest may include a subregion of a cardiac surface up to an entire cardiac surface for which the signals have been measured or reconstructed. In one example, the computed signal features can include cardiac rhythm of the respective signals, including signals measured responsive to a respective temporary intervention and a reference signals in the absence of any intervention. The feature calculator 66 further can be configured to evaluate the detected rhythm over multiple heartbeats (e.g., as defined by beat detector 62) to ascertain whether the computed signal features described a normal sinus rhythm or an arrhythmogenic condition. Additionally or alternatively, the feature calculator 66 can be programmed to compute a cycle length and respective frequency components for the respective heartbeat intervals that have been identified. The respective computed features can be stored as part of the EP data 28 (e.g., as feature metadata) or otherwise associated (linked) with the EP data 28. Additionally, feature calculator 66 can be programmed to derive one or more conduction patterns from local bipolar or unipolar electrograms as respective features. As an example, the sequence of activation from the distal to the proximal electrode in a multipolar catheter can be used to describe a beat and determine a change with respect to an existing beat/pattern (e.g., a variation from a baseline beat/pattern).

The control function 40 can be programmed to control the signal processing function 44 and/or the output generator 42 based on analysis of the computed features and/or parameters from maps generated by the reconstruction engine 46. For example, the data analysis function 60 can analyze the computed signal features over multiple heartbeat intervals, such as by computing statistics (e.g., mean or variance) associated with such features. In response, the control function 40 can trigger the output generator 42 to generate a corresponding electrocardiographic map on a surface of interest or multiple surfaces based on the cardiac EP data 28 for respective heartbeat intervals that include only the computed signal features. Additionally or alternatively, the control function 40 can trigger the output generator 42 to generate a corresponding electrocardiographic map on a surface of interest or multiple surfaces based on the cardiac EP data 28 for a continuous time interval that includes or encompasses the set of computed signal features. The generation of the maps and/or results of such signal processing can be displayed in the foreground and displayed to the user responsive to the control function 40. Alternatively, the control function can cause the map generation and/or signal processing to be implemented by respective functions running as background processes, and can be selected in response to a user input (e.g., selecting a radio button or other graphical user interface element) and rendered on the display.

As another example, the control function 40 can trigger the output generator 42 to generate a corresponding electrocardiographic map on one or more surfaces of interest in response to detecting changes in one or more signal parameters and/or signal features. In one example, the changes can be detected based on determining a variation in such parameters and/or features with respect to a baseline. The baseline can be generated in response to a user input specifying a normal or baseline signal waveform from which the baseline parameter(s) and/or feature(s) are derived. Alternatively, the baseline can be identified automatically based on analysis of measured EP signals over an extended time period for a given patient relative to known signals for the patient and/or a patient population. Still further the baseline can be determined based on measurements obtained at the start of or during an early phase of an intervention (e.g., prior to ablating tissue). The baseline can represent one or more signal parameters and/or features determined for a normal cardiac rhythm or a cardiac arrhythmia (e.g., a baseline AT or baseline PVC). The control function 40 thus can trigger the output generator 42 to generate a respective map and/or signal processing 26, 44 in response to detecting changes in one or more such baseline parameters and/or features.

In another example, the changes in one or more signal parameters and/or signal features can be detected based on comparing one or more such parameters and/or features relative to one or more respective thresholds. The threshold value can be determined as a percentage change (e.g., a relative threshold) from a prior value of such parameter. Alternatively, the threshold can be a threshold value specifying an absolute value of a feature, parameter or a value representative of a change in such feature or parameter. Each such threshold can be implemented as a default value or be set in response to a user input. Thus, the threshold can be set for a patient cohort or be patient specific. In some examples, to trigger the output generator 42 to automatically generate a map or perform automated signal processing, as described herein, the change can include a combination of more than one change, such as can be defined as by combinatorial logic stored in memory and executed by the control function 40.

As a further example, the signal processing 44 can include an application of one or more signal processing functions 26, 44 to the EP data 28 in response to the analysis of the computed signal features and/or parameters. In an example, the control function 40 can apply signal processing 44 to process the EP data 28 that is stored in memory. In another example, the control function 40 can configure the signal processing 26 of the signal measurement device 18 to adjust the signal processing that is applied to the signals measured from the respective electrodes 14 and/or 16. The signal processing function 26, 44 can include one or more of identification of bad channels, filtering (e.g., notch filter, band pass filter, low pass filter or the like).

Additionally, as described herein, the control function 40 can trigger the reconstruction engine 46 and/or the output generator 42 to generate respective graphical maps of reconstructed EP signals for the surface of interest or a portion thereof responsive to the data analysis 60 of the computed signal features and/or parameters. For example, the output generator 42 can be programmed to generate a graphical map that spatially includes one or more regions of interest up to and including the entire surface (e.g., an entire cardiac surface) for one or more time intervals.

In one example, the time interval for which the map is generated includes one or more heartbeat intervals for which the data analysis function 60 has detected a change in the computed signal features. For example, in response to detecting changes in the cardiac rhythm, the control function 40 can trigger the output generator 42 to generate one or more graphical maps to be provided to the display 36. In some examples, a dialog box can pop up to allow the user to accept or reject the display of proposed graphical map that has been generated. Additionally, the dialog box can include further information about the map, such as including a description of the type of map and the detected signal features that have triggered the map to be generated (e.g., based on the feature metadata). In this way a user can be provided with actionable information more quickly and with fewer manual user input steps. The system may also display a comparison of the two rhythms, for example through subtraction of common elements, in order to identify regions with modified or differing activity.

As a further example, the control function 40 includes instructions programmed to compare the generated map with respect to a reference map and to identify regions of interest and/or differences between the automatically generated map and the reference map based on the comparison. The reference map can be generated for the surface of interest (e.g., the same region as the map being compared) based on cardiac electrophysiological signals for one or more intervals, such as from measurements acquired prior to those used to trigger automatic map generation (e.g., from baseline patient data).

In another example, the data analysis 60 can determine changes in cycle length signal features. In response to detecting such changes in cycle length (e.g., compared to a local or global cycle length threshold), the control function 40 can activate the output generator 42 to provide one or more graphical maps for a region of interest or up to the entire surface of interest responsive to the detected cycle length changes. For example, the detected cycle length changes can be indicative of an arrhythmogenic condition (e.g., atrial fibrillation or other arrhythmia) or a change from an arrhythmia to a normal condition or from a normal cycle to arrhythmia, and the computed signal features can be used to automatically generate a map containing information relevant to the detected condition. As mentioned, a pop-up dialog box can be generated to alert the user that a corresponding map has been generated in response to such cycle length changes, which can be accepted or rejected by the user in response to a user input through the user interface 48. The dialog can also provide information describing the type of signal changes as well as specify a location on a cardiac surface where such changes occurred (e.g., based on feature metadata).

In some examples, in response to the control function 40 detecting stable cardiac activity over a time interval (e.g., for at least a predetermined duration), such as a stable rhythm or stable cycle length over a period of multiple heartbeats determined based on computed signal features (e.g., by feature calculator 66), the control function 40 can invoke the signal processing function 26, 44 to automatically perform certain signal processing functions. For example, the control function 40 can invoke the signal processing function 26 and/or 44 to improve signal quality, such as by implementing signal averaging across a plurality of heartbeat intervals for each of the respective signals. In response to the condition changing from a stable cardiac signal to an unstable condition, the signal averaging (or other signal quality improving function) can be terminated and an additional action taken, such as described above.

In another example, the feature calculator 66 is programmed to apply a trained machine learning (ML) model to the one or more parameters determined (e.g., by EP data analyzer 64) to determine respective signal features of the EP signals for a portion of a time interval. For example, the ML model (e.g., machine-readable instructions executable by a processor) can be pre-trained to automatically detect and classify one or more types signal features, such as a normal rhythm or arrhythmias, such as tachycardia, bradycardia, atrial fibrillation or others. Signal waveforms classified as normal further can be used to determine baseline signal features and/or parameters, which can be stored in memory for the given patient and used to determine variations with respect to the baseline, as described herein.

In some examples, the system 10 can be used during an interventional procedure, such as including delivery of energy (e.g., ablation or electrical stimulation, such as pacing or cardiac resynchronization therapy (CRT)) and/or application of a bioactive agent (e.g., chemical or pharmaceutical and the like). The delivery of energy and/or application of a bioactive agent can be adapted to effect a change in tissue function, which can be temporary or permanent according to the objective of the intervention. For example, when a physician is performing ablation, the mapping system 30 can provide feedback to help drive respective ablations (or other interventions). Traditionally, in labs that perform EP, feedback for ablation is obtained using 12-lead ECG and intracardiac catheters; however such traditional approaches are often insufficient to detect meaningful changes in real-time for complex arrhythmias, such as persistent atrial fibrillation. The mapping system 30 can provide enhanced real-time feedback for procedures, such as ablation, CRT or the like, to increase efficacy and improve outcomes.

For example, the mapping system 30 can be configured to provide communication among pacing, ablation and/or other interventions and real-time mapping (e.g., electrocardiographic imaging (ECGI)). The non-invasive component of the real-time mapping can further be programmed to detect and determine when a given ablation occurs and automatically analyze cardiac signals and maps derived from such signal before and after the given ablation. Additionally, the data analysis 60 can be programmed to determine whether one or more signal features (e.g., computed by feature calculator 66) for a region of interest of the heart have changed in response to the given intervention and, as disclosed herein, automatically generate an ECGI map to display information about the change, which can be rendered as a graphical map 34 on display 36.

For example, the EP data analyzer 64 is programmed to extract parameters from the EP signals (e.g., measured or reconstructed EP signal). The feature calculator 66 can compute features based on such signal parameters. In an example, the feature calculator can compute such features from the parameters by applying an ML model trained to compute one or more features, including signal morphology, cycle length, dominant frequency, earliest activation region, slowest conduction region, and conduction patterns (e.g., focal and reentrant) as well as changes in such features. If the data analysis detects changes in the electrophysiological condition for one or more regions of interest responsive to the intervention (e.g., ablation, stimulation, application of bioactive agent, etc.), the data analysis 64 can tag the respective region(s) to indicate that the region under intervention is part of a critical circuit path or source sustaining the arrhythmia, and thus may warrant additional ablation or other intervention in the region. If no such changes are observed responsive to the ablation, the mapping and navigation system can suggest another region of interest or indicate the need to ablate at the same region with an increased power or duration.

By way of example, prior to applying an intervention, the output generator 421 can identify an initial region of interest as a localized target on or within the patient’s heart based on one or more of the data analysis function 60 applied to a map of that is generated (by reconstruction engine 46) based on the EP data 28 and geometry data 38. One or more initial target regions can be identified based on the data analysis function 60 in the absence of applying an intervention. For example, the initial target(s) can be locations on the cardiac surface identified as including irregular cardiac activity, such as one or more rotors, focal points or containing bursting cycle length or fractionization of signals across a cardiac surface. Thus, a user can move the interventional device to a selected one of the target regions for applying an intervention to alter the electrical properties of the selected target region. The navigation system 22 can provide guidance to enable the user to position the interventional device 19 for applying the intervention. Additionally, or alternatively, an imaging modality (e.g., x-ray, ultrasound etc.) can be used for positioning the interventional device 19 at the desired target location.

The output generator 42 is programmed to generate electroanatomic maps for the surface of interest based on EP data 28 acquired over time that includes multiple time intervals, in which at least one of the time intervals (a first time interval) occurs during or after (e.g., responsive to) application of a given intervention to temporarily perturb electrical properties of a region of interest on or within the patient’s heart. As described herein, the interventional device can be configured to apply the intervention directly or indirectly to the region of interest by controlling delivery of an energy and/or a bioactive agent to induce or inhibit conduction of electrical signals for the region of interest. In some examples, the interventional device 19 is configured to apply the first intervention as a non-lethal intervention to alter the electrical properties of cardiac tissue in the region of interest temporarily for one or more cardiac cycles during the first time interval based on a first control instruction provided by the control system 21. The control instruction can be generated in response to a user input to the interventional control system 21 or be based on a command issued by the control 40.

Another electroanatomic map is generated (e.g., by reconstruction engine 46) based on EP data 28 acquired during at least one other time interval in the absence of the first (e.g., temporary) intervention. This other time interval can occur before or after the first time interval. In one example, the EP data 28 is acquired for the other time interval during application of a different intervention to the region of interest, which can be a different temporary intervention or a permanent intervention. In another example, the EP data 28 is acquired for the other time interval in the absence of applying any intervention, which can be at a time prior to applying any intervention (e.g., baseline EP data from the same or a prior EP study) or at a time after applying an intervention. The other map thus can be generated based on the EP data 28 acquired during such other time interval to provide another set of data to evaluate the efficacy of the first intervention. Accordingly, the resulting map can provide a visualization of cardiac electrophysiological signals to demonstrate cardiac activity for the patient as a comparative example to the map generated based on EP data acquired during application of the first (e.g., temporary) intervention.

In some examples, the data analysis function 60 is configured to determine changes (e.g., by computing a difference) between a set of EP data 28 acquired during the first intervention and another set of EP data acquired at a time after the first intervention. The time after the intervention can be a default time (e.g., fixed time) or it can be programmable in response to a user input (e.g., via user interface 48), such as ranging from a time immediately after (e.g., less than one second after) the first intervention is removed or up to several seconds after or minutes after removing the first intervention. The data analysis function 60 can be configured to capture the other set of EP data at the designated time and determine corresponding changes automatically responsive to the first intervention being removed or terminated. The output generator 42 can further generate output data 32 to provide a respective graphical map to visualize the determined changes on the display 36. In this way, user can be provided information automatically based on which the user can better understand the effect of the first intervention without manually having to manually configure and control what information is included in the graphical map. As a result, the application of intervention and resulting patient care can be facilitated.

As a further example, the other EP data 28 represents a baseline measure of electrical activity to which the corresponding EP data acquired during the intervention can be compared. For example, the EP data 28 representing the baseline measure of electrical activity includes one or more of electrocardiogram data (e.g., from a 12 lead ECG), body surface electrical data (e.g., acquired by 50 or more body surface electrodes), reconstructed electrical data (e.g., reconstructed from body surface EP data and geometry data) and/or invasively measured electrical data (e.g., acquired by sensors associated with a probe or catheter used to provide an intervention). In this example, the currently acquired EP data 28, which is acquired by the same approach as the baseline data, can provide feedback that the data analysis function 60 compares to baseline data to determine a change (e.g., a delta) between the baseline and current measurements. This delta can be evaluated over time while the intervention (e.g., a temporary or permanent intervention) is being applied to control the intervention. For example, the control function 40 is configured to apply the intervention so long as the delta (determined by the data analysis function 60) continues to vary during the intervention. In response, to the delta computed intermittently during the intervention, the control function can generate instructions to the output generator 42 to issue a notification (e.g., graphical, textual and/or audible) to inform the user of the impact of the intervention. In response to being notified that the delta is no longer changing, the control function 40 can issue a command to automatically terminate application of the intervention. Alternatively, the user can manually terminate the intervention responsive to the notification. The manner in which the notification is provided can vary. In one example, the notification can be one color code on a GUI element to indicate a positive impact (e.g., therapeutic effect) by the intervention, such as while the delta exceeds a threshold or the delta continue to change over time. Another color can be used to indicate when no sufficient positive impact is being caused by the intervention, such as when the delta is below a threshold or the delta is no longer changing in response to the intervention.

In some examples, the data analysis function 60 can control a frequency at which delta is computed from the EP data acquired for computing the delta depending on the type of intervention, including different types of temporary or permanent interventions. The type of intervention can be determined automatically based on information received from an interface to which the interventional device 19 and/or control system 21 are coupled. Additionally, or alternatively, the type of intervention can be determined in response to a user input (e.g., data entered at user interface 48 through the user device 50 to specify the type of intervention). The control function 40 can also use the determined type of intervention to control the interventional device, such as described herein. As an example, the frequency at which delta is computed can be set to first time interval (e.g., every 0.5 to 1.0 seconds) for RF ablation or PF ablation, a second longer duration (e.g., 4 to 10 seconds) for cyroablation, or an even longer for various types of chemical interventions that can be applied. In this way, the data analysis and control functions 60 and 40 can appropriately evaluate the impact and implement functions to visualize and control application of various different types of interventions.

The data analysis function 60 is further programmed to analyze the respective maps generated based on the EP data, according to any of the functions disclosed herein, and determine respective changes in the maps or changes in information derived from the maps (e.g., by EP data analyzer or feature calculator 66) generated for the respective two more time intervals. In one example, the data analysis function determines the change represent a positive therapeutic effect being achieved responsive to applying the first intervention. An example of a positive therapeutic effect includes determining a change in rhythm in one map toward an optimal rhythm (e.g., less arrhythmic) in the other map generated responsive to the intervention. Other examples of positive effects responsive to applying an intervention that can be ascertained based on evaluating respective maps include reduced number or frequency of rotors and/or foci, reduced levels of fractionization, reduced bursting, and the like. Other reductions in arrhythmogenic activity or arrhythmia drivers can also be determined by the data analysis function 60.

In another example, determined changes between respective maps can represent a negative therapeutic effect (e.g., an increase in arrhythmia) or a lack of change responsive to applying the first intervention. The output generator 42 can generate guidance based on the determined changes indicating whether or not a desired effect is achieved responsive to the first intervention. In response to determining a positive therapeutic effect for a given temporary intervention, the output generator can inform the user that a permanent intervention can be applied to the same target site where the given temporary intervention was applied. This can be used to apply a second intervention to permanently alter electrical properties of the region of interest based on the analysis function 60 determining a desired change in cardiac rhythm condition responsive to the first intervention changes. For example, the control system 21 can be programmed to control interventional device 19 to set a level of the energy and/or a potency (e.g., a cytotoxic potency) of the bioactive agent for the intervention, such as based on control instructions provided by the control 40 and/or responsive to a user input. In a further example, the control 40 can be programmed to provide control instructions to the control system 21 for controlling the interventional device 19 to apply a permanent intervention, such as ablation or a cytotoxic agent to the target region of interest based on previously applied temporary intervention at the target region of interest.

Alternatively, if the determined change responsive to the first intervention indicates a negative or lack of desired therapeutic effect being achieved responsive to the first intervention, the output generator 42 can display a recommendation on the display 36 to select a next target region of interest for another application of the first (non-lethal) intervention based on such determined change. The next target region of interest can be identified based on the analysis function 60 or from a previously generated list of potential targets.

In some examples, the output generator 42 can also be programmed to generate a report that describes steps performed in the EP procedure by the user. The report can include data described in each of the steps implemented by the user in response to the respective user input as well as data describing the computed signal feature/characteristics and/or changes thereof associated with the cardiac EP signals over relevant time intervals. The report can also include information describing sites where each intervention is applied and respective intervention data (e.g., type of intervention, intervention parameters, time etc.) The acceptance or rejection of automated maps or signal processing functions applied by the control function 40 can also be stored in memory and included as part of the report to document information presented to the user during the procedure. The reports can be stored in memory and/or sent to one or more other users (e.g., by email, text messaging or other messaging protocol) for further review/analysis and/or archiving thereof.

FIG. 2 depicts an example of the EP data analyzer 64. The EP data analyzer 64 is configured to generate signal parameters 108 based on EP data 28 which can include measured EP data, reconstructed EP data or a combination of measured or reconstructed EP data. The EP data analyzer includes a signal feature extractor 104 and a parameter generator 106. The signal feature extractor 104 is configured to analyze one or more respective EP signals and identify corresponding signal characteristics within one or more heartbeat intervals of the respective signals. For example, signal features can include changes in local or global cardiac cycle length, changes in activation patterns, changes in signal morphology, changes in frequency components and/or any other modifications to the electrical characteristics of the region of interest. An example of such a change could be the termination of atrial fibrillation to an atrial tachycardia, having both a slower cycle length and one that is consistent across the chamber/region of interest. The parameter generator 106 is configured to process the extracted features and to generate the signal parameters 108. Thus, as described herein, the signal parameters characterize different attributes of the respective signals, such as may include a value representative of respective signal features including amplitude (e.g., a normalized amplitude), a frequency or period, as well as corresponding parameters for respective waveform components of the respective heartbeat intervals. The features of such waveform components (e.g., the Q, R, S, T and/or P waveform components) may include morphological as well as other respective segments and/or intervals of the waveform components within a respective heartbeat or between sequential beats. The resulting signal parameters 108 can be stored in memory for further processing by the feature calculator function 66, as described herein.

FIG. 3 depicts an example of the feature calculator function 66, which receives as respective inputs the signal parameters 108 as well as the EP data 28 and/or 102. The feature calculator 66 includes a rhythm calculator 120, a cycle length calculator and a machine learning (ML) model 124. The ML model 124 can be trained to determine one or more categories of signal features from the heartbeat intervals based on the signal parameters 108 that have been determined. The categories of signal features can include normal cardiac rhythm and one or more arrhythmia conditions.

For example, the ML model 124 can include any of an artificial neural network (ANN) algorithm, a support-vector machine (SVM) algorithm, a decision tree algorithm, a recurrent neural network (RNN) algorithm, and a convolutional neural network (CNN) algorithm. Other types of machine learning can be used in other examples. The ML model 124 can be trained on prior EP signals measured invasively and/or non-invasively from a patient population representative of one or more known categories of signal features during a respective time interval or over a series of time intervals. Additionally or alternatively, the ML model 124 can be trained on prior EP signal reconstructed (e.g., by respective instances of reconstruction engine 46) on to a respective surface of interest from a patient population representative of one or more known categories of signal features. In a further example, one or more ML models 124 can be configured to process resulting graphical maps, consistent with how the ML model 124 is trained to identify respective trained categories of arrhythmias or a normal rhythm. As a further example, the ML model 124 can be configured to evaluate respective input maps and label respective maps that are highly correlated (e.g., similar) to known training data to classify respective signals across a surface of interest into one or more categories, which can include a normal cardiac rhythm or one or more types of arrhythmias. The input maps can be a set of one or more maps generated by output generator 42 based on the EP data 28 and geometry data 38 for a patient over one or more time intervals.

The feature calculator 66 can store the determined signal features in memory. A feature analyzer 126 can be programmed to analyze the determined features over time to determine an indication of changes over time. For example, the feature analyzer 126 can determine a change between successive heartbeat intervals or over a larger period of time, which includes a time interval during or otherwise responsive to application of a given intervention and another time interval without the given intervention. As another example, the feature analyzer 126 can determine a rate of change for a respective feature over time such as by analyzing a plurality of heartbeat intervals and a respective feature thereof. The feature analyzer 126 can provide the determined indication of changes in the respective features to the control function 40. As described herein, the control function 40 can control the output generator and/or the intervention control system based on the determined changes. The control function 40 can also trigger the signal processing function 44 and/or the output generator 42 in response to the analysis of the determined features provided by the feature calculator 66. The control function 40 could also trigger an update to the geometry data 38 based on anatomical changes such as due to a change in intracardiac pressure, volume changes or other structural changes that could occur during a procedure. The changes in geometry data 38 further could trigger output generator 42 to regenerate a map to reflect the structural changes that are detected. This can include generate a map for another time interval during application of an intervention applied at the same target site with different parameters or at a different site.

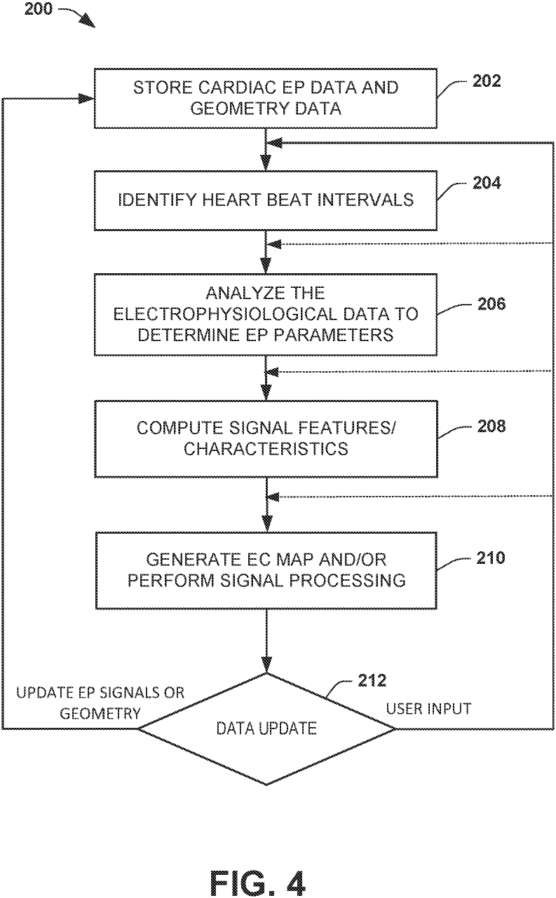

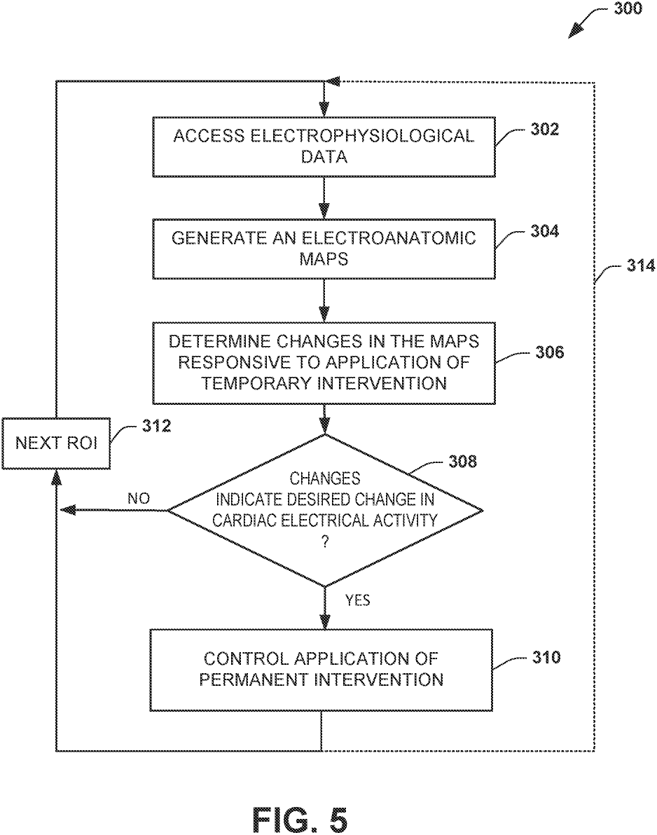

FIGS. 4 and 5 depict examples of methods 200 and 300 such that can be implemented by the system 10 to perform respective functions herein. While for purposes of simplicity of explanation, the example method of FIG. 4 is shown and described as executing serially, the example method 200 is not limited by the illustrated order, as some actions could in other examples occur in different orders, multiple times and/or concurrently from that shown and described herein. Additionally, each of the methods 200 and 300 can be implemented as machine-readable instructions executed by a processor, such as by the mapping system 30. Accordingly, the description of FIG. 4 and also refer to FIGS. 1-3.

Referring to FIG. 4, the method 200 begins at 202 in which cardiac EP data and geometry data are stored (e.g., EP data 28 and geometry data 38 are acquired and stored in memory). At 204, respective heartbeat intervals are identified (e.g., by beat detector 62), such as based on EP data representative of cardiac EP signals measured over a time interval. The identified beats can be used to tag some or all of the EP data 28 to specify one or beats for further analysis.

At 206, the method includes analyzing a portion of the EP data (e.g., as defined by a plurality of the respective heartbeat intervals) to determine one or more parameters associated with the cardiac EP signals over the time interval. In an example, the heartbeat intervals can be detected automatically and stored as beat data with the EP data to specify the detected heartbeat intervals for the respective cardiac electrophysiological signals. The EP signals on the surface of interest, as represented in the EP data, can include respective EP signals measured invasively from the surface of interest and/or measured non-invasively from an outer body surface. In another example, additionally or alternatively, EP signals on the surface of interest, as represented in the EP data, includes reconstructed electrophysiological signals, which are calculated for the surface of interest by solving the inverse problem based on non-invasively measured electrophysiological signals and/or invasively measured signals.

At 208, the method includes computing signal features associated with the cardiac electrophysiological signals over at least a portion of the time interval based on the one or more parameters. In an example, the signal features are computed (e.g., by feature calculator 66) based on changes in respective signal features over at least a portion of the time interval based on analyzing the one or more parameters for at least two samples over time interval. For example, a graphical map can be generated (e.g., by output generator 42) responsive to the computed changes in the signal features indicating an instability and/or an arrhythmogenic condition. Alternatively, automated signal processing can be performed (e.g., by signal processing function 26 and 44) responsive to the computed changes in the signal features indicating a stable rhythm and/or a non-arrhythmogenic condition. In some examples, the signal features associated with the cardiac electrophysiological signals can be computed by applying a trained ML model to the one or more parameters determined for the portion of the electrophysiological data to ascertain the respective signal features associated with the cardiac EP signals. As described herein, the signal can include a cardiac rhythm and/or cycle length for electrophysiological signals distributed across a surface of interest.

At 210, the method includes generating a map on a surface of interest and/or performing automated signal processing based on the cardiac electrophysiological signals for heartbeat intervals that include the computed signal features (at 208). As described herein, the map can be generated automatically responsive to computing the signal features and/or the other automated signal processing can be performed automatically responsive to computing the signal features. The automated signal processing at 210 can include one or more of identifying one or more bad measurement channels, applying signal filtering or performing inverse reconstruction of electrophysiological signals on the surface of interest based on the electrophysiological data.

At 212, the method determines whether any data has been updated. If the EP signals (e.g., measured and/or reconstructed) have been updated, the method returns to 202 to repeat the method. In response to receiving a user input, in which instructions or commands are updated to control one or more aspects of the method 200, the method returns to preceding part of the method to repeat the remaining portions. The location to where the method returns can vary depending on the user input instruction that is received (as shown by dotted lines in FIG. 4).

In some examples, the method 200 can also include generating a report summarizing steps performed in the method. The report thus can include data describing each of the computed signal features and/or changes thereof associated with the cardiac electrophysiological signals over at least a portion of the time interval. The report data can be stored in memory.

The method 300 of FIG. 5 can be implemented to evaluate the impact of an intervention and control subsequent interventions. The method 300 includes accessing electrophysiological data 28 representing cardiac electrophysiological signals measured from a patient’s body 12. For example, the EP data 28 can be measured non-invasively by an arrangement of body surface electrodes 16 on an outer surface of the patient’s body and/or invasively by one or more electrodes within the patient’s body. At 304, electroanatomic maps is generated for the signals over multiple time intervals. The time intervals can be consecutive heartbeats or the heartbeats can be spaced apart in time. For example, the reconstruction engine 46 is programmed to reconstruct electrophysiological signals on locations distributed across the surface of interest within the patient’s body to provide the map based on the EP data and geometry data 38 for one or more time intervals, in which the surface of interest includes a region of interest. In an example, a portion of the time intervals occurs during or after a first intervention, which is applied (e.g., by interventional device 19) to temporarily perturb electrical properties of the region of interest on or within the patient’s heart. The region of interest can be identified, initially, as a localized target on or within the patient’s heart based on analysis of electrophysiological signals (e.g., by data analysis function 60) for a surface of interest on another map, which can include measured and/or reconstructed signals in the absence of applying an intervention.

At 306, the method 300 includes determining changes in the map or information derived from the map responsive to application of the intervention. For example, the changes in the map or information derived from the map can include or be based on the data analysis and signal features implemented by the method 200 of FIG. 4. For example, the analysis is performed on electroanatomic map for the surface of interest (e.g., a cardiac surface) by comparing analysis results and/or signal features for multiple maps to ascertain how the application of the localized intervention affects the cardiac electrical activity. As described herein, the intervention can include delivery of non-lethal energy and/or a bioactive agent to induce or inhibit conduction of electrical activity for the region of interest for a transitory duration. The intervention can be applied directly or indirectly (e.g., outside of the region of interest) to the region of interest to alter temporarily the electrical properties of cardiac tissue in the region of interest for one or more cardiac cycles.

At 308, a determination is thus made to ascertain whether the changes determined at 306 indicate a desired (or intended) change in cardiac electrical activity. If the determination at 308 is positive (“YES”), indicating a desired change in cardiac electrical activity responsive to the first intervention, the method proceeds to 310. At 310, the method includes controlling a second intervention to permanently alter the electrical properties of the region of interest. For example, interventional control system 21 can control the interventional device 19 to permanently alter the electrical properties of at least a portion of cardiac tissue in the region of interest. For instance, the control system 21 can control a level of the energy and/or a cytotoxic potency of the bioactive agent, such as in response to instructions provided by the control function 40.

From 310, the proceeds to 312. Also, if the determination at 308 is negative (“NO”), based on the determined changes indicating a negative or lack of desired therapeutic effect responsive to the first intervention, the method proceeds to 312. At 312, a next region of interest is identified for another application of the temporary intervention. For example, guidance can be generated by output generator to identify the next target region of interest to the user (e.g., on display 36), such as a localized target identified on or within the patient’s heart. The next target region of interest can be selected based on evaluating the estimated electrophysiological signals in the map (e.g., by data analysis function) responsive to applying the first intervention. Alternatively, the next location can be selected by the user, such in response to a user input at user device 50 or from a list potential regions of interest. From 312 the method returns to 302 to repeat 302-308. If a temporary intervention applied to the next region of interest results in changes in the cardiac electrical activity, indicating a desired effect is achieved responsive to such intervention, the method can proceed to 310 to control the application of another intervention to permanently alter the electrical properties of the second region of interest. The method can be terminated by a user at any time, such as after the cardiac electrical activity sufficiently improves. Additionally, in some examples, the method can proceed from 310 directly to 302 (shown by dotted line 314) to generate a map following application of the permanent intervention, which can be evaluated at 302-308 to ensure that the desired therapeutic effect has been achieved by application of the permanent intervention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A method comprising:
applying a first intervention to perturb electrical properties of a region of interest on or within a patient’s heart during a perturbation interval that includes at least a portion of one or more cardiac cycles;
generating, by a computing device comprising a processor, an electroanatomic map for a surface of interest based on cardiac electrophysiological data representing cardiac electrophysiological signals over a time interval that includes the perturbation interval;
evaluating, by the computing device, the map for at least the time interval to determine changes in cardiac electrical activity responsive to the first intervention; and
controlling a second intervention to permanently alter the electrical properties of the region of interest based on the determined changes in the cardiac electrical activity.

2. The method of claim 1, wherein applying the first intervention includes applying non-lethal energy and/or a bioactive agent to induce or inhibit conduction of electrical signals for the region of interest.

3. The method of claim 1, wherein the first intervention is applied directly to the region of interest.

4. The method of claim 1, wherein the first intervention is applied outside of the region of interest.

5. The method of claim 1, wherein applying the first intervention includes applying an energy and/or a bioactive agent to alter temporarily electrical properties of cardiac tissue in the region of interest.

6. The method of claim 5, wherein controlling the second intervention includes controlling the energy and/or the bioactive agent to permanently alter the electrical properties of at least a portion of the cardiac tissue in the region of interest.

7. The method of claim 6, wherein controlling the second intervention further comprises controlling a level of the energy and/or a cytotoxic potency of the bioactive agent based on the map or information derived from the map indicating a desired change in rhythm condition responsive to the first intervention.

8. The method of claim 7, wherein the second intervention includes ablation applied to the region of interest.

9. The method of claim 5, wherein the first intervention includes applying one of electrical stimulation, cryomapping or reversible electroporation.

10. The method of claim 1, further comprising generating guidance based on the determined changes in the cardiac electrical activity indicating whether a desired effect is achieved responsive to the first intervention.

11. The method of claim 10, wherein generating the guidance includes setting a parameter to control the applying of the second intervention at the region of interest based on the determined changes representing a positive therapeutic effect being achieved responsive to the first intervention.

12. The method of claim 10, wherein generating the guidance includes displaying a recommendation to identify a next target region of interest for another application of the first intervention based on the determined changes representing a negative or lack of desired therapeutic effect being achieved responsive to the first intervention.

13. The method of claim 1, wherein prior to applying the first intervention, the method comprises identifying the region of interest as a localized target identified on or within the patient’s heart in another map that is generated based on electrophysiological signals measured in the absence of applying an intervention to perturb the electrical properties of the region of interest.

14. The method of claim 1, wherein the region of interest includes a first region of interest and a second region of interest on or within the patient’s heart, wherein prior to applying the second intervention, the method comprises:
identifying the second region of interest as a localized target identified on or within the patient’s heart based on evaluating electrophysiological signals or another map measured responsive to applying the first intervention; and
controlling the second intervention to permanently alter the electrical properties of the second region of interest.

15. The method of claim 1, further comprising:
measuring the cardiac electrophysiological signals non-invasively by an arrangement of body surface electrodes on an outer surface of a patient’s body; and
reconstructing electrophysiological signals on locations distributed across the surface of interest within the patient’s body to provide an electroanatomic map based on the measured cardiac electrophysiological signals and geometry data, in which the surface of interest includes at least the region of interest.

16. The method of claim 1, further comprising:
measuring the cardiac electrophysiological signals invasively by one or more electrodes within a patient’s body to provide at least a portion of the electrophysiological data.

17. A system, comprising:
non-transitory memory configured to store data and machine-readable instructions, the data including electrophysiological data representing cardiac electrophysiological signals for a plurality of locations across a cardiac surface over time;
one or more processors adapted to access the memory and execute the instructions programmed to perform a method comprising:
generating an electroanatomic map for the cardiac surface based on the electrophysiological data acquired over time that includes a first time interval and at least one other time interval, one of the first or other time intervals occurring during or after application of a first intervention to temporarily perturb electrical properties of a region of interest on or within a patient’s heart;
determining changes in the map or in information derived from the map between the first time interval and the other time interval; and
controlling a second intervention to permanently alter the electrical properties of the region of interest based on the determined changes.

18. The system of claim 17, further comprising an interventional device configured to deliver an energy and/or a bioactive agent to induce or inhibit conduction of electrical signals for the region of interest.

19. The system of claim 18, wherein the interventional device is configured to set a level of the energy and/or a potency of the bioactive agent based on control instructions provided by the one or more processors.

20. The system of claim 18, wherein the interventional device is configured to apply the first intervention directly or indirectly to the region of interest.

21. The system of claim 18, wherein the interventional device is configured to apply the first intervention to alter temporarily electrical properties of cardiac tissue in the region of interest for at least one cardiac cycle during the first time interval based on a first control instruction provided by the one or more processors.

22. The system of claim 21, wherein the one or more processors are further programmed to provide a second control instruction based on the map or information derived from the map indicating a desired change in a rhythm condition responsive to the first intervention, and the interventional device is configured to set a level of applied energy and/or a cytotoxic potency of the bioactive agent delivered by the interventional device during the second intervention based on the second control instruction.

23. The system of claim 18, wherein the interventional device is a first interventional device, the system further comprising a second interventional device configured to apply the energy or the bioactive agent to permanently alter the electrical properties of the region of interest.

24. The system of claim 18, wherein the interventional device is configured to apply one of electrical stimulation, cryomapping or reversible electroporation.

25. The system of claim 18, wherein the one or more processors are further programmed to generate guidance based on the determined changes indicating whether a desired effect is achieved responsive to the first intervention.

26. The system of claim 25, wherein the one or more processors are further programmed to set a parameter to control the interventional device for the application of the second intervention based on the determined changes representing a positive therapeutic effect being achieved responsive to the first intervention.

27. The system of claim 25, wherein the one or more processors are further programmed to display a recommendation to identify a next target region of interest for another application of the first intervention based on the determined changes representing a negative or lack of desired therapeutic effect being achieved responsive to the first intervention.

28. The system of claim 17, wherein prior to the application of the first intervention, the one or more processors are further programmed to generate an other map based on electrophysiological signals measured in the absence of any application of an intervention to perturb the electrical properties of the region of interest, and to identify the region of interest as a localized target identified on or within the patient’s heart based on the other map.

29. The system of claim 17, wherein the region of interest includes a first region of interest and a second region of interest on or within the patient’s heart, wherein prior to the application of the first intervention, the one or more processors are further programmed to:
identify the second region of interest as a localized target identified on or within the patient’s heart the map that is generated based on evaluating electrophysiological signals measured responsive to the application of the first intervention; and
control the application of the second intervention to permanently alter the electrical properties of the second region of interest.

30. The system of claim 17, further comprising an
an arrangement of body surface electrodes adapted to measure the cardiac electrophysiological signals non-invasively from an outer surface of a patient’s body, the one or more processors further programmed to reconstruct electrophysiological signals on locations distributed across a surface of interest within the patient’s body to provide the map based on the measured cardiac electrophysiological signals and geometry data, in which the surface of interest includes at least the region of interest.

31. The system of claim 17, further comprising:
one or more electrodes adapted to measure the cardiac electrophysiological signals invasively within a patient’s body.

32. The system of claim 17, wherein the other time interval occurs before the the first time interval.

33. A computer implemented method, comprising:
accessing, by a computing device comprising a processor, electrophysiological data representing cardiac electrophysiological signals measured from a patient’s body; and
generating an electroanatomic map for a surface of interest based on the electrophysiological data acquired over time that includes a first time interval and at least one other time interval, one of the first or other time intervals occurring during or after application of a first intervention to temporarily perturb electrical properties of a region of interest on or within the patient’s heart;
determining changes in the map or information derived from the map responsive to application of the first intervention, the first intervention including delivery of non-lethal energy and/or a bioactive agent to induce or inhibit conduction of electrical activity for the region of interest; and
controlling a second intervention to permanently alter the electrical properties of the region of interest based on the determination indicating a desired change in cardiac electrical activity responsive to the first intervention.
